(19) United States
(12) Patent Application Publication
(10) Pub. No.: US 2009/0143840 A1
(43) Pub. Date: Jun. 4, 2009

(54) AUDITORY PROSTHESIS UTILIZING INTRA-NEURAL STIMULATION OF THE AUDITORY NERVE

(76) Inventors: John C. Middlebrooks, Ann Arbor, MI (US); Russell L. Snyder, Logan, UT (US)

Correspondence Address: Casimir Jones, S.C., 440 Science Drive, Suite 203, Madison, WI, 53711, US

(21) Appl. No.: 12/278,382

(22) PCT Filed: Feb. 6, 2007
(86) PCT No.: PCT/US07/02912

§ 371 Date: Jan. 21, 2009

Related U.S. Application Data
(60) Provisional application No. 60/765,620, filed on Feb. 6, 2006.

(51) Int. Cl. A61N 1/36

(52) U.S. Cl. 607/57

(57) ABSTRACT

The present invention relates to auditory prostheses. In particular, the present invention provides an auditory prosthesis capable of direct, intra-neural stimulation of the auditory nerve.

This invention claims priority to U.S. Provisional Patent Application No. 60/765,620 filed Feb. 6, 2006, hereby incorporated by reference in its entirety.

This invention was made with government support under contract number NO1-DC-5-0005 awarded by the National Institute on Deafness and Other Communication Disorders (NIDCD). The government has certain rights in the invention.

### FIELD OF THE INVENTION

The present invention relates to auditory prostheses. In particular, the present invention provides an auditory prosthesis capable of direct, intra-neural stimulation of the auditory nerve.

### BACKGROUND OF THE INVENTION

Approximately 5 to 10% of the population suffer from impaired hearing. Various degrees of deafness exist, for example, ranging from mild, to moderate, to severe, to profound. Deafness can be acquired or congenital deafness. The cause for such hearing losses can lie in the region of the ear which conducts the sound wave (e.g., ear drum, middle ear), in the inner ear (e.g., cochlea), or in the auditory nerve or central auditory processing. Depending upon the cause, site, and degree of hearing difficulty, operative therapy, rehabilitation, drug therapy, or other therapies may be indicated. When these therapies are insufficient or unsuccessful, there are a variety of technical devices (e.g., hearing aids and auditory prostheses) available in order to improve and/or restore hearing.

Heretofore, conventional cochlear implants (e.g., generally consisting of an array of electrodes placed in the scala tympani of the cochlea), have existed as one means of stimulating the auditory nerve. Electrical stimulation of the structures of the cochlea leads to activity in the auditory pathway of the brain, leading to a sensation of hearing.

However, the position of a scala-tympani electrode array, in a volume of electrically conductive perilymph, located at a variable distance from the osseous spiral lamina, and separated from auditory nerve fibers by a bony wall, results in multiple indirect, attenuated current paths from stimulated electrodes to nerve fibers. The lack of direct access to auditory nerve fibers imposes multiple limitations including high threshold levels for stimulation, imprecise frequency activation, a limited number of independent information channels from the ear to the brain, activation of non-contiguous tonotopically inappropriate cochlear locations and limited frequencies of stimulation.

Thus, there is a need for an auditory prosthesis that overcomes one or more of these as and other limitations that exist with regard to currently available auditory prostheses.

### DESCRIPTION OF THE DRAWINGS

FIG. 1 shows one embodiment of a stimulating array of the present invention, a 16-site thin-film silicon-substrate stimulating array.

FIG. 2 shows one approach used to insert the intra-neural stimulating array. The upper panel (A) shows a post-mortem dissection of a cat's ear, viewed roughly orthogonal to the cochlear round window (center), which was exposed by making a hole in the lateral wall of the bulla, an expansion of the cat's middle ear cavity. The round window membrane has been removed from the round window, but the round window margin is otherwise intact. The basilar membrane of the basal half of basal turn can be seen at the black arrow as a dark crescent. The parallel arc of the spiral ganglion can be seen as a dark line in the osseous spiral lamina (white arrow). The white filled circle indicates the location of the hole in the bone of the osseous spiral lamina through which an intra-neural silicon array can be inserted. The lower panel (B) shows a silicon array inserted into the modiolar trunk of the auditory nerve through an opening in the osseous spiral lamina. The round window margin was enlarged to permit this array placement. The dashed line indicates the location of the round-window margin prior to enlargement.

FIG. 3 shows (A) plots characteristic frequencies of recorded neurons as a function of depth in the inferior colliculus and (B-H) spatial tuning curves (STCs) recorded from the inferior colliculus in response to acoustic tones presented during normal-hearing conditions. The contours in each of panels B to H represent responses to tones of a particular frequency, indicated in each panel. The vertical dimension of the plots represents depth in the inferior colliculus and the horizontal dimension represents the sound level. Tones at low sound levels activated relatively narrow regions of the colliculus. At higher levels these tones activated broader regions of the colliculus more strongly. Successive increases in tone frequency resulted in shifts of STCs to progressively deeper locations in the inferior colliculus. The STCs illustrate natural activation of the colliculus with the highest selectivity.

FIG. 4 shows spatial tuning curves (STCs) evoked by monopolar stimulation using a conventional banded intra-scalar cochlear implant. Panels A and C indicate responses to individual stimulation of cochlear-implant channels MP3 and MP8, respectively. Panel B indicates the response to simultaneous stimulation of channels MP3 and MP8 at stimulus levels below the threshold for activation by either channel alone. The relatively poor selectivity of stimulation using a conventional cochlear implant is readily apparent. Electrodes 3 and 8 evoke activity across nearly half the depth of the colliculus traversed by the recording probe.

FIG. 5 shows STCs elicited by stimulation using the intra-neural silicon-substrate electrode array. Stimuli were presented through 8 of the 16 implanted stimulation sites. The relatively high degree of stimulus selectivity in most of these STCs (A, C, D, E, G and H) is typical of intra-neural stimulation.

FIG. 6 shows STCs elicited by stimulation using the 6 sites of the intra-neural silicon-substrate electrode array. As in FIG. 5, most of the activity in these STCs show markedly greater selectivity than that observed following stimulation with conventional cochlear implant electrodes.

FIG. 7 shows the distribution among recording sites of the spread of excitation elicited by acoustic tones (labeled Tone), intra-neural stimulation (IN), bipolar stimulation with a conventional cochlear implant (BP), and monopolar stimulation with a conventional cochlear implant (MP). In this “box and whisker” plot, the bottom, middle, and top horizontal lines on each box represent the 25^{th}, 50^{th}, and 75^{th} percentile of the distribution, the whiskers represent 1.5 times the interquartile distance, and the plus signs represent outlying data points. The number printed over each set of box and whiskers indicates the number of tone frequencies (for IN) or electrical stimulation sites (for IN, BP, and MP) that are represented in each distribution. Panels A, B, and C indicate activation at levels 3, 6, and 10 dB above threshold. This figure allows these three forms of auditory prosthesis stimulation to be quantitatively compared.

FIG. 8 shows STCs elicited by intra-neural stimulation using individual channels (Panels A, C, and E) or simultaneously by pairs of channels (B, D, and F) as indicated by the lines with arrowheads. In each case the simultaneous paired stimulation evokes activity that is the sum of that activated by each channel alone indicating that there is little interaction between the stimuli on each channel.

FIG. 9 illustrates a scatter plot of Single-Electrode Threshold Difference, a measure of the overlap of active neural populations, on the horizontal axis and Threshold Reduction, a measure of the reduction in activation threshold resulting from simultaneous stimulation on the vertical. Lower amounts of threshold reduction represent lower amounts of between-channel interference. These plots demonstrate that interference among simultaneously stimulated channels is greater for conventional cochlear implant stimulation (upper panel) than for intra-neural stimulation (lower panel).

FIG. 10 shows a photograph of a human temporal bone from a cadaver. This is the medial aspect, viewed from the inside of the cranium. Several possible sites of auditory nerve stimulation are indicated with numbers. This view illustrates the locations along the nerve that would be stimulated, not the actual approaches. The four sites are named by their associated surgical approaches: (1) intracranial; (2) infra-labyrinthine; (3) juxta-cochlear; and (4) intra-modiolar.

FIG. 11 shows a photograph of a human temporal bone from a cadaver. This is the lateral aspect, viewed from the side, showing three possible sites for insertion of an intra-neural stimulating array. The bone of the mastoid process has been removed so that the middle ear space can be seen. The round window membrane has been removed so that the osseous spiral lamina can be seen inside the round window. The intra-modiolar approach can be through a small hole placed in the osseous spiral lamina. The temporal bone below the vestibular labyrinths has been removed to expose the auditory nerve with the square at left. The inset at the lower left shows the auditory nerve exposed using the infra-labyrinthine approach. The nerve is seen just lateral to the auditory meatus at a location. The circle indicates the location of the juxta-cochlear access.

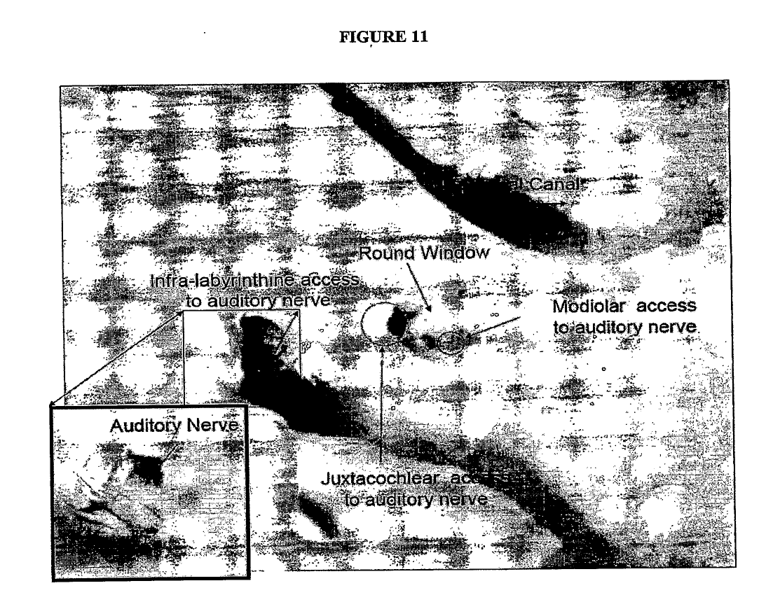

FIG. 12 shows a photograph of a human temporal bone from a cadaver. This is the lateral aspect, shown at higher magnification than in FIG. 11. The locations of access to the auditory nerve using the juxta-cochlear and intra-modiolar approaches are labeled.

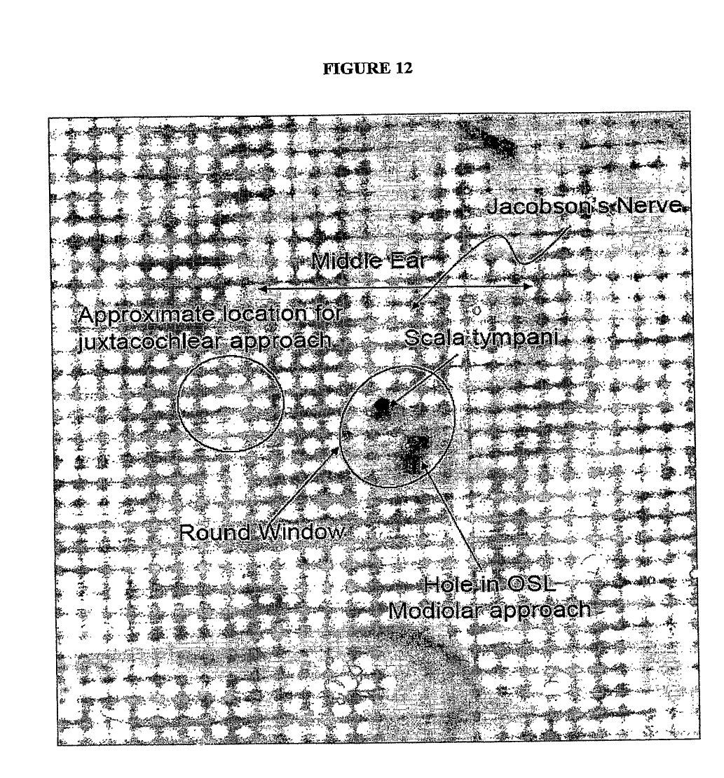

### DETAILED DESCRIPTION OF THE INVENTION

Hearing aids and auditory prosthetics have been based on one of two basically different principles: acoustic mechanical stimulation, or electrical stimulation. With acoustic mechanical stimulation, sound is amplified in various ways and delivered to the inner ear as mechanical energy. This may be through the column of air to the ear drum, or direct delivery to the ossicles of the middle ear. Acoustic mechanical stimulation generally requires that the structure of the cochlea, hair cells, the auditory nerve, and the central processing centers all be intact. The more hair cells that are destroyed or not functioning properly, the less effective acoustic mechanical stimulation can be.

Electrical stimulation functions differently. With this method, used when the structures of the cochlea (e.g., the hair cells) are disrupted, the sound wave is transformed into an electrical signal (e.g., by a cochlear implant). The electrical stimulation produced by the cochlear implant leads to activation of the auditory nerve leading to activation of the auditory pathway of the brain and a sensation of hearing. Electrical stimulation does not require that the structure of the cochlea and the hair cells be intact. Rather, a sufficiently intact auditory nerve and central processing centers suffice. In currently available cochlear implants, the stimulating electrodes (e.g., that generate electrical stimulation) are placed within the scala tympani of the cochlea as close as possible to the nerve endings of the auditory nerve.

Electrode arrays of currently available cochlear implants are placed in the scala-tympani at some distance from auditory nerve fibers. Implantation of an electrode array at this position, in a volume of electrically conductive perilymph, located at a variable distance from the osseous spiral lamina, and separated from auditory nerve fibers by a bony wall, has its drawbacks. For example, stimulation provided by arrays at this position results in multiple indirect, attenuated current paths from stimulated electrodes to nerve fibers. Furthermore, the lack of direct access to auditory nerve fibers imposes additional limitations. These limitations include the fact that thresholds for stimulation (e.g., current levels important for neural stimulation) with scala-tympani electrodes are relatively high, tonotopic spread of activation by a scala-tympani electrode is broad (e.g., often more broad than the response to a one-octave noise band), a broad spread of activation by scala-tympani electrodes results in interactions among activated neural populations, thereby limiting the number of independent information channels, scala-tympani electrodes can produce ectopic activation of auditory nerve fibers (e.g., activation of fibers in non-contiguous, tonotopically inappropriate cochlear locations), currently available scala-tympani arrays reach only to the middle of the second cochlear turn (e.g., well short of the apical regions representing the lowest frequencies), and in cases of meningitis, bacterial labyrinthitis, and otosclerosis, the scala tympani of the basal turn may be occluded, rendering placement of scala-tympani electrode arrays difficult or impossible.

Thus, there is a need for an auditory prosthesis that overcomes limitations that exist with regard to currently available auditory prostheses.

Accordingly, the present invention provides an auditory prosthesis capable of direct, intra-neural stimulation of the auditory nerve. In some embodiments, the auditory prosthesis comprises electrodes positioned directly in the auditory nerve trunk. Thus, in some preferred embodiments, the present invention provides an auditory prosthesis that provides direct, intra-neural stimulation (e.g., via direct electrical stimulation (e.g., via electrodes) of the modiolus or auditory nerve (e.g., the auditory nerve trunk)).

Although an understanding of the mechanism is not necessary to practice the present invention and the present invention is not limited to any particular mechanism of action, in some embodiments, direct, intra-neural stimulation (e.g., via electrodes positioned directly in the modiolus or auditory nerve trunk) addresses (e.g., reduces and/or eliminates) one or more drawbacks mentioned herein regarding conventional intra-scalar stimulation. For example, direct intra-neural stimulation provides thresholds of stimulation that are lower (e.g., in some embodiments, 10 decibels (dB) lower, in some embodiments, 15 dB lower, in some embodiments, 20 dB lower, in some embodiments, 25 dB lower, in some embodiments, 30 dB or more lower) than that of stimulation with scala-tympani electrodes. For example, experiments conducted during development of the present invention revealed intra-neural stimulation thresholds that averaged 24.5 dB lower than monopolar (MP) scala-tympani stimulation and that averaged 34.1 dB lower than biopolar (BP) scala-tympani stimulation (See, e.g., Example 4).

Furthermore, intra-neural electrode based stimulation produces more restricted tonotopic spread of activation compared to activation by a scala-tympani electrode (See, e.g., Examples 3 and 4). The tonotopic spread of activation by a scala-tympani electrode is broad, often broader than the response to a one-octave noise band (See, e.g., Example 3). In contract, intra-neural electrodes produce more restricted activation (e.g., at near-threshold current levels as measured by spatial tuning curves (STCs); See, e.g., Example 4). Thus, the present invention provides an auditory prosthesis that possesses more restricted (e.g., that is lower and/or narrower) activation patterns and lower tonotopic spread of activation compared to conventional cochlear implant devices. Although an understanding of the mechanism is not necessary to practice the present invention and the present invention is not limited to any particular mechanism of action, in some embodiments, the more restricted activation patterns and lower tonotopic spread provided by an auditory prosthesis of the present invention provides a subject using such a device a quality of hearing not attainable with heretofore available auditory prostheses (e.g., such a subject may experience a greater number and/or higher quality of independent information channels (e.g., due to more refined activation of neural populations) than experienced by a user of a conventional prosthesis).

In some embodiments, the present invention provides an auditory prosthesis that overcomes the broad spread of activation by scala-tympani electrodes (e.g., that results in interactions among activated neural populations, thereby limiting the number of independent information channels). For example, an auditory prosthesis of the present invention provides direct access of intra-neural electrodes to more-restricted neural populations. Although an understanding of the mechanism is not necessary to practice the present invention and the present invention is not limited to any particular mechanism of action, in some embodiments, such direct access results in reduced channel interactions and a larger number of effectively independent information channels (e.g., compared to conventional cochlear implant devices).

Experiments conducted during the development of the present invention indicated monopolar stimulation of basal cochlear sites with conventional scala-tympani electrodes resulted in undesirable ectopic activation of intra-modiolar fibers passing from the cochlear apex (e.g., activation of non-contiguous, tonotopically inappropriate cochlear locations). In some embodiments, an auditory prosthesis of the present invention (e.g., comprising intra-neural electrodes) produces less ectopic activation (e.g., at a variety of current levels (e.g., low, medium, and high).

In some embodiments, an auditory prosthesis of the present invention stimulates (e.g., via direct electrical stimulation via an electrode) auditory nerve fibers originating from throughout the spiral ganglion. Although an understanding of the mechanism is not necessary to practice the present invention and the present invention is not limited to any particular mechanism of action, in some embodiments, this results in activation of portions of the auditory pathway representing the entire range of normal hearing, whereas conventional prosthesis electrodes activate primarily basal (high frequency) fibers. In some embodiments, an auditory prosthesis of the present invention (e.g., comprising intra-neural electrode arrays) is used in situations in which the scala tympani of the basal turn of a subject is occluded (e.g., in cases of meningitis, bacterial labyrinthitis, and otosclerosis).

In some embodiments, an auditory prosthesis of the present invention stimulates (e.g., via direct electrical stimulation via an electrode) apical regions (e.g., representing frequencies less than ˜1 kHz) of the inferior colliculus.

In some embodiments, the intra-neural stimulation is provided via an array of electrodes. For example, in some embodiments, a 16-site silicon-substrate stimulating probe is used (See Middlebrooks and Snyder, JARO, in press, 2007). In some embodiments, current levels (e.g., levels of electrical stimulation) needed for neural activation using an auditory prosthesis of the present invention are lower than the current levels required for the same level of neural activation using a conventional cochlear implant device. In some embodiments, reduced thresholds of activation offer extended battery life (e.g., used to generate electrical stimulation).

Tonotopically specific stimulation with scala-tympani electrodes was limited to the basal half of the cochlea. In contrast, intra-neural stimulation produced activation of restricted loci distributed across the entire cochlear spiral (e.g., corresponding to frequencies from below 500 Hz up to 32 kHz and beyond). Thus, in some embodiments, the present invention provides an auditory prosthesis capable of activating auditory nerve fiber populations originating from restricted sites distributed throughout the entire cochlear spiral (e.g., wherein the activation corresponds to frequencies ranging from below 500 Hz up to 32 kHz and beyond).

In some embodiments, an auditory prosthesis of the present invention comprises a 16-channel isolated current source. In some embodiments, the present invention provides stimulation software (e.g., configured for use with a 16 channel stimulator).

Thus, the present invention provides an auditory prosthesis comprising intra-neural electrodes (e.g., positioned directly in the modiolus or auditory nerve trunk) that overcomes one or more existing limitations of conventional cochlear implants. Intra-neural stimulating arrays overcome obstacles encountered in patients in whom the scala tympani is occluded by bone, such as in a victim of meningitis or severe otosclerosis. However, it is also contemplated that the intra-neural stimulating array may become a favored alternative to the intrascalar implant even for patients for whom the intra-scalar device is possible. For example, access to the entire frequency range, which is afforded via use of an intra-neural stimulation device of the present invention, offers enhanced low frequency hearing, thereby improving perception of spoken and musical pitch and perhaps enhanced spatial hearing. In some embodiments, a patient with partial residual hearing might favor an intra-neural array (e.g., because it can be inserted into the nerve, this is an approach likely to have minimal effect on residual hearing). Additionally, more-precise tonotopic activation provided by a device of the present invention can enhance transmission of spectral information (e.g., improving speech reception in noise, vertical and front-back sound localization, and recognition of musical timbre). The reduced thresholds also offers extended battery life for external stimulators and in some embodiments, it is contemplated to be a totally implantable device needing no external battery pack. Additionally, intra-neural stimulation provided by a device and/or system of the present invention provides an increase in the number of independent channels of information that can be transmitted through the auditory prosthesis. Speech tests in present-day cochlear-implant users suggest that they benefit from no more than 6-8 channels of information even though a scala-tympani array might contain as many as 24 electrodes. The reduced between-channel interference demonstrated with intraneural stimulation provides that, in some embodiments, an increase in the number of independent channels will be perceived by a subject using a device and/or system of the present invention (e.g., leading to enhanced speech recognition in noise and other improvements and benefits in prosthetic hearing).

### EXPERIMENTAL

The following examples are provided in order to demonstrate and further illustrate certain preferred embodiments and aspects of the present invention and are not to be construed as limiting the scope thereof.

### Example 1

### Materials and Methods

Experiments were conducted in barbiturate-anesthetized cats. Responses to acoustic tones, to electrical stimulation with a conventional cochlear implant, and to electrical stimulation with an intra-neural array were characterized. Neural activity was recorded from the inferior colliculus of the midbrain as a means of monitoring activation of the ascending auditory pathway. The right ear was deafened by disarticulation of the ossicles. The right inferior colliculus was visualized by aspiration of overlying occipital cortex. A 32 channel, silicon-substrate recording probe was inserted through the inferior colliculus oriented in the coronal plane and angled from dorsolateral to ventromedial at an angle of 45° from the mid-sagittal plane. This trajectory allowed the probe to span up to 6 octaves of the tonotopic organization of the colliculus from below 500 Hz to above 32 kHz, which is most of the normal range of hearing in the cat. The probe had 32 recording sites (400 μm in area) positioned on a single shank at 100 μm intervals. Neural waveforms were recorded simultaneously from all 32 sites and saved to computer disk. On-line peak picking and graphic display permitted continuous monitoring of responses. Off-line spike sorting allowed examination of isolated single unit and multi-unit cluster activity.

Each experiment began with testing of responses to acoustic stimulation in normal-hearing conditions. Calibrated noise- and tone-burst stimuli were presented through a hollow ear bar to the left ear. The position of the recording probe was adjusted based on responses to sounds, then the brain surface was covered with agarose and the probe was fixed in place with acrylic cement. Measurements of frequency tuning provided a functional measure of the location of each recording site along the tonotopic axis.

After completion of tests with acoustic stimuli, the left cochlea was deafened by intra-scalar injection of neomycin sulfate and a conventional cochlear implant array was implanted in the scala tympani. This cochlear implant was an 8-electrode animal version of the NUCLEUS24 device from Cochlear Corp. The dimensions were identical to the distal 8 electrodes of the human device: platinum band electrodes, 400 μm in diameter, centered at 750 μm intervals along a silastic carrier. Electrical stimuli through the cochlear implant consisted of single biphasic pulses, 40 or 200 μs per phase, initially cathodic. Stimuli were presented in monopolar (MP) and bipolar (BP) electrode configurations.

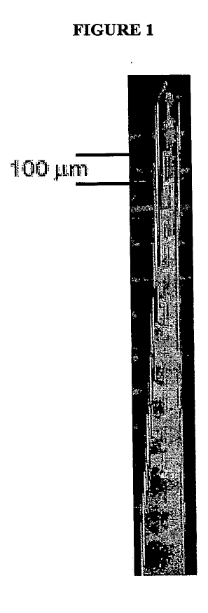

Testing of the scala-tympani electrode was followed by testing of intra-neural stimulation. The intra-neural array was a 16-site thin-film silicon-substrate array (See FIG. 1). The sites were positioned at 100 μm intervals along a single shank. Stimuli were biphasic pulses, 40 or 200 μs per phase, initially cathodic, presented in a MP configuration.

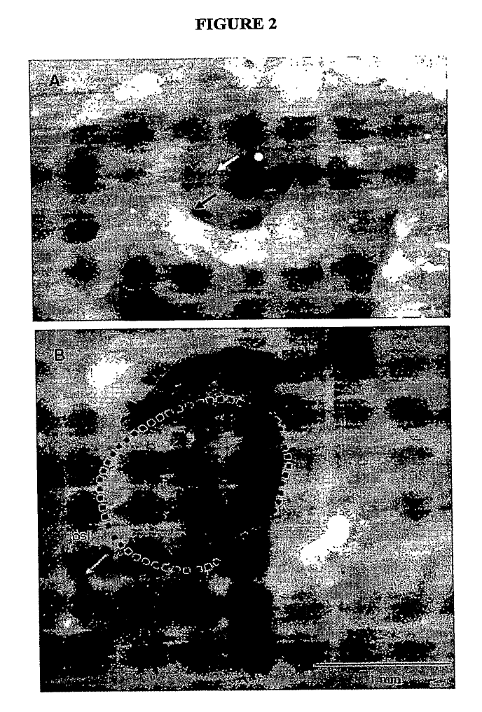

The intra-neural electrode array was positioned as follows. The left bulla was opened to expose the cochlea. The round-window membrane was excised and the rim of the round-window was enlarged with a diamond burr. The beveled tip of a 26-gauge needle was used to make an opening in the osseous spiral lamina below the spiral ganglion. The hole was enlarged with a fine reamer. The probe was inserted under visual control using a micromanipulator. Several orientations of the stimulating array were tested. In some embodiments, one successful orientation was approximately in the coronal plane, from ventrolateral to dorsomedial, approximately 45° from the horizontal plane. The array insertion point in a post-mortem dissection is shown in FIG. 2A. The black arrow indicates the location of the basilar membrane. The white arrow indicates the location of the spiral ganglion. The white circle indicates a site on the osseous spiral lamina at which a hole could be made to insert an intra-neural stimulating array. The array is shown in position for stimulation in an intra-operative photo in FIG. 2B.

### Example 2

### Responses to Acoustic Stimulation

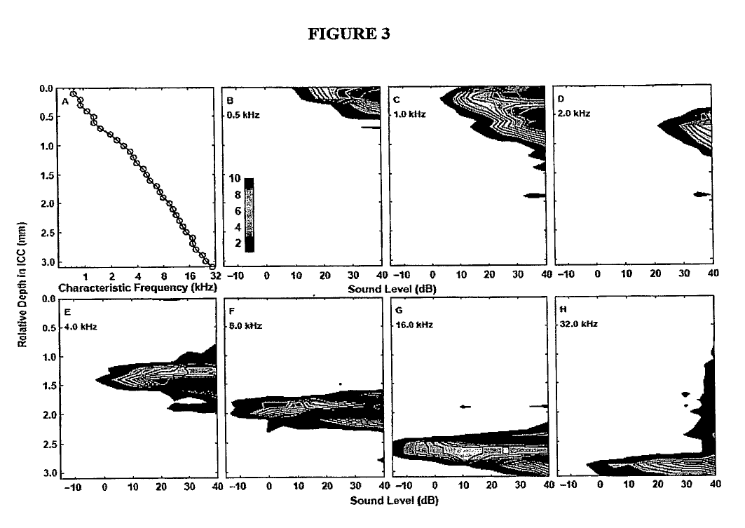

Responses to acoustical tones were used to identify the positions of recording sites relative to the tonotopic axis of the inferior colliculus and to characterize the spread of excitation by tones under normal-hearing conditions. The frequency tuning of responses to tones was similar to those commonly reported in the inferior colliculus. The tonotopic progression of characteristic frequencies (CFs) as a function of the relative depth in the IC (distance along the shank of the recording probe; See FIG. 3A) was consistent with the commonly reported tonotopic organization of the inferior colliculus. Responses to tones under normal-hearing conditions are shown in FIG. 3. Each of the panels B through H represents responses to tones at a particular frequency as indicated in each panel. Responses are shown in the form of Spatial Tuning Curves (STCs). In each STC, the vertical dimension represents depth in the inferior colliculus and the horizontal dimension represents sound level. The contours represent cumulative discrimination index, which is a measure of the magnitude of the response. The vertical extent of the contours in each panel represents the spread of above-threshold activation in the inferior colliculus in response to a particular frequency.

### Example 3

### Inferior Colliculus Responses to Conventional Intra-Scalar Stimulation

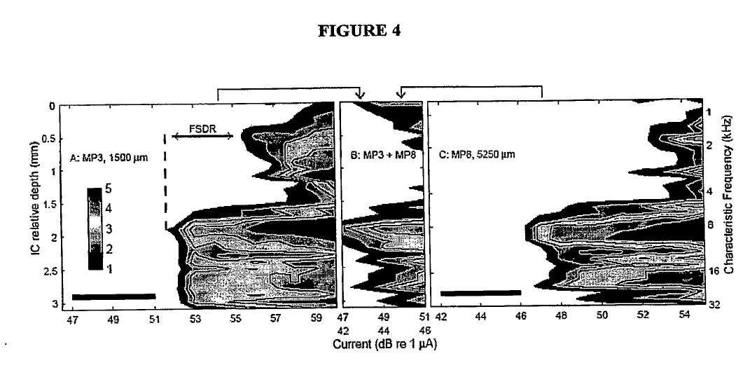

Following recordings in normal-hearing conditions, the left cochlea was deafened, a conventional scala-tympani electrode array was implanted, and inferior colliculus responses to scala-tympani stimulation were recorded. Scala-tympani stimulation in the MP configuration produced broad activation of recording sites spanning the tonotopic axis. In FIGS. 4A and C, STCs show responses to monopolar (MP) stimulation through individual cochlear implant channels, MP3 (See FIG. 4A) and MP8 (See FIG. 4C). Stimulation of the most apical sites of this array even at the lowest current levels activated recording probe sites broadly distributed throughout the deepest half of the inferior colliculus, representing the high frequency basal cochlea. At stimulation levels only about 2 to 4 dB higher, neural activation spread to encompass the entire tonotopic axis of the inferior colliculus, including the representation of apical cochlear sites well away from any of the scala-tympani electrodes. The activation of the apical representation indicates spread of excitation to intra-modiolar apical fibers passing the basal scala-tympani electrodes.

### Example 4

### Inferior Colliculus Responses to Intra-Neural Stimulation

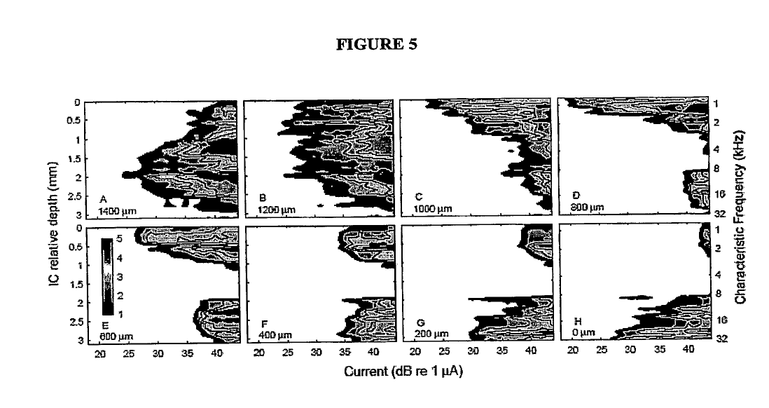

Single biphasic electrical pulses (40 μs/phase) were presented through a silicon-substrate electrode array inserted in the modiolar portion of the auditory nerve. FIG. 5 shows STCs representing the responses recorded from the inferior colliculus to individual stimulation of 8 of 16 intra-neural electrodes. Individual intra-neural electrodes activated auditory nerve fibers corresponding to the lowest (e.g., FIG. 5D) and highest (e.g., FIG. 5H) frequencies represented in the inferior colliculus. In many instances, stimulation of a single intra-neural electrode activated a single discrete region in the inferior colliculus (See, e.g., FIGS. 5A, C-E, and H). In other instances, a single intra-neural electrode activated two discrete regions (See, e.g., FIG. 5F). Thresholds for intra-neural stimulation averaged 24.5 dB lower than for intra-scalar stimulation in the same animals.

The topography of intra-neural stimulation reflected the spiral geometry of auditory nerve fibers within the modiolus. Low frequency fibers from the apical turn (which are mapped superficially in the inferior colliculus) are found in the center of the intra-modiolar nerve trunk, overlaid first by middle-turn fibers, and then, most peripherally, by high frequency fibers from the cochlear base (mapped to the deep inferior colliculus). Correspondingly, stimulation of the deepest intra-neural electrode, located somewhat past the center of the nerve (See, e.g., FIG. 5A), activated the middle frequency representation in the inferior colliculus. Successively more superficial electrode sites activated progressively lower frequency representations (See, e.g., FIG. 5D) and then higher frequency representations (See, e.g., FIG. 5H).

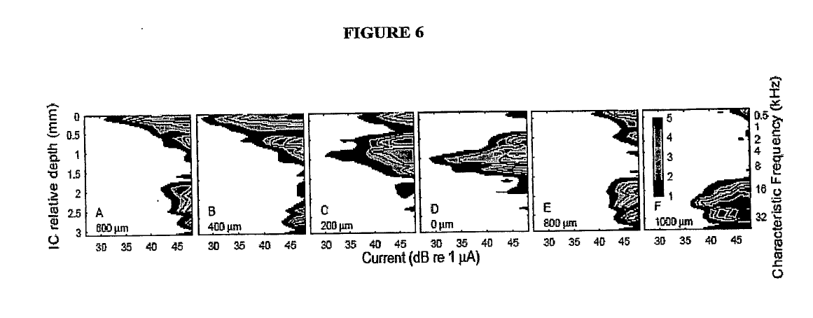

Additional examples of spatial tuning curves from stimulation using an intra-neural arrays are shown in FIG. 6. In the example shown in FIG. 6, the panels have been sorted by a automatic computer algorithm according to the location of activity in the inferior colliculus. In this way, intra-neural stimulation channels could be selected to activate a progression from low- to high-frequency regions of the auditory nerve.

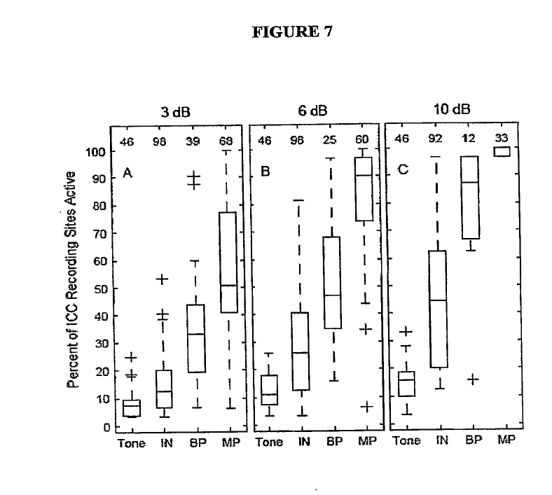

The spread of excitation elicited by intra-neural stimulation was more restricted than that elicited by stimulation with a conventional cochlear implant. FIG. 7 represents the distribution among multiple tonal frequencies and stimulation sites resulting from stimulation with acoustic tones (labeled Tone) and from electrical stimulation using intra-neural stimulation (labeled IN), bipolar cochlear implant stimulation (labeled BP), and monopolar cochlear implant stimulation (labeled MP). Panels A, B, and C show the distributions at 3, 6, and 10 dB above the threshold for each stimulation condition, respectively. Intra-neural stimulation consistently produced more restricted spread of excitation than did monopolar cochlear implant or bipolar cochlear implant stimulation.

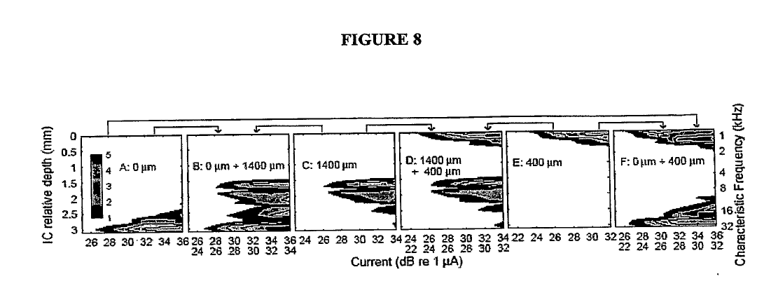

In addition to more restricted activation, simultaneous stimulation of pairs of intra-neural electrodes resulted in substantially less interference between electrodes than did simultaneous stimulation of pairs of cochlear implant electrodes. FIG. 8 shows STCs representing responses to stimulation of 3 individual intra-neural electrodes (in panels A, C, and E) and STCs representing responses to simultaneous stimulation of 3 pairs of intra-neural electrodes (in panels B, D, and F). In each pair-wise stimulation condition, the contribution of each individual electrode is evident and there is little or no influence of one electrode on the threshold for stimulation of the other electrode.

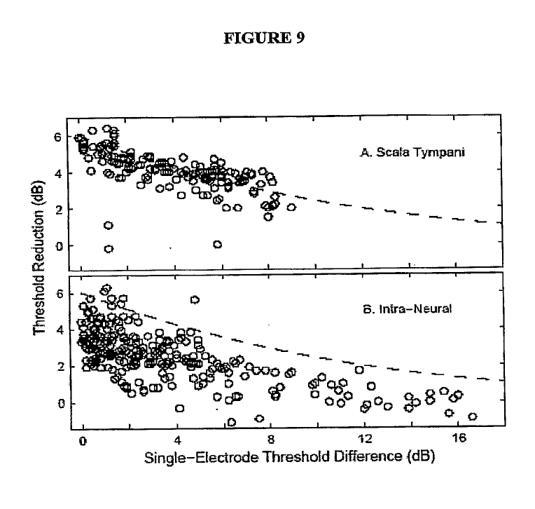

FIG. 9 shows a measure of the interference between pairs of electrodes stimulated simultaneously. Panels A and B show data from scala tympani and intra-scalar electrodes, respectively. Data are drawn from multiple inferior colliculus recording sites. The horizontal dimension of each panel shows the Single-Electrode Threshold Difference, which is a measure of the overlap of inferior-colliculus regions activated by individual stimulation of the two electrodes in each tested pair. The presence of data points extending to higher values in Panel B indicates that there was less overlap for intra-neural than for intra-scalar stimulation. The vertical dimension of each panel shows the Threshold Reduction, which is a measure of the amount by which stimulation of one electrode in a pair interferes with the threshold of the other electrode in the pair. That measure generally was lower in the intra-neural case, indicating that interference among simultaneously stimulated electrodes was less for intra-neural stimulation than for cochlear implant stimulation.

The results shown above for intra-neural stimulation were obtained using a lateral approach to the auditory nerve (e.g., one embodiment of which is illustrated in FIG. 2). In other experiments, an intra-cranial approach to the auditory nerve was tested. In those tests the nerve was approached from the posterior cranial fossa, and the intra-neural stimulating array was positioned into the auditory nerve as it exited the medial end of the internal acoustic canal, the internal meatus. In those experiments, spread of excitation generally was broader and the topography of stimulation of various frequency representations was less consistent among repeated intra-cranial array placements than was the case using the lateral approach. In addition, there is concern that in an application in human patients, pulsation of the intra-cranial portion of the auditory nerve relative to a stimulating array may result in damage to the auditory nerve. For these reasons, the intra-cranial approach to the auditory nerve is regarded as less than optimal for placement of an intra-neural stimulating array.

### Example 5

### Surgical Approaches for Implantation of Intra-Neural Stimulating Arrays Evaluated in Human Cadaver Temporal Bones

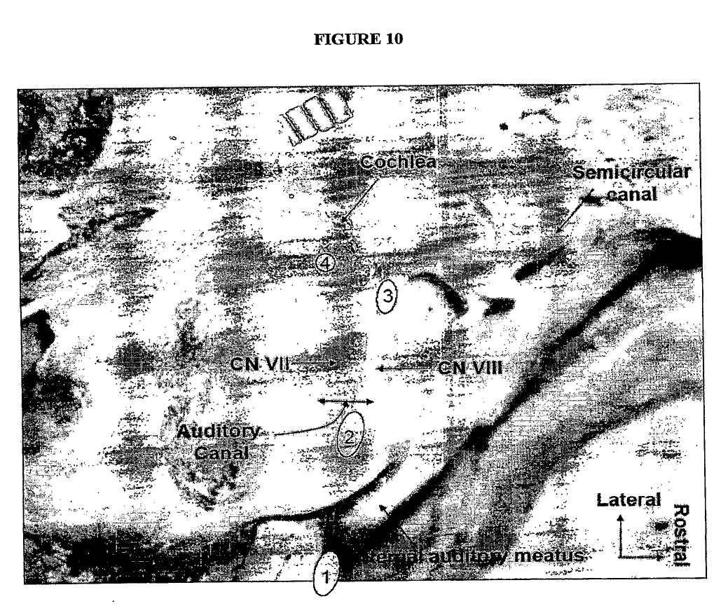

Approaches to the auditory nerve were evaluated in dissections of human post-mortem (cadaver) material. The first approach that was evaluated was an intra-cranial approach by way of the posterior fossa. This is represented by site #1 in FIG. 10. The intracranial approach offers direct visualization of the 8th nerve with little or no drilling on the temporal bone and its attendant effects (e.g., potentially deleterious) on residual hearing. However, this approach requires opening the posterior fossa, the negative sequellae of loss of CSF, possible infections of meninges, damage to the facial nerve, and vascular spasm of the blood supply to the cochlea. In addition, inserting the prosthetic electrode array into and fixing the prosthesis within the pulsating, free floating nerve at this location may present problems.

The infra-labyrinthine approach allows the nerve to be accessed within the more confined space of the medial internal auditory canal, but CSF loss, nerve pulsations and vascular spasm are still judged to be significant problems. Moreover, it was regarded as less than optimal because in many instances access to the nerve using this approach may be blocked by the jugular bulb.

In the juxta-cochlear approach the nerve can be directly visualized, CSF loss and vascular spasm are judged to be minimal, and direct damage to the cochlea is also minimal.

In some embodiments, one advantage of the intra-cranial, infra-labyrinthine, and juxta-cochlear approach is that they can be employed with the least compromise of residual hearing.

The intra-modiolar approach is a direct approach that allows visualization of the nerve, albeit somewhat limited, with minimal loss of CSF and minimal possibility of infection. This surgical approach is similar to the standard surgical “facial recess” approach for conventional cochlear implants and is therefore familiar to most otologists. The intra-modiolar approach is analogous to the approach that has been evaluated physiologically in the animal model described above in Examples 1-4. Thus, in some preferred embodiments, the intra-modiolar approach is utilized for placement of a device of the present invention.

All publications and patents mentioned in the above specification are herein incorporated by reference. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

What is claimed is:

1. An auditory prosthesis comprising intra-neural electrodes, wherein said electrodes are configured for positioning directly in the modiolus or auditory nerve trunk.

2. The auditory prosthesis of claim 1, wherein said electrodes stimulate the auditory nerve.

3. The auditory prosthesis of claim 1, wherein direct intra-neural stimulation provides thresholds of stimulation that are lower than stimulation thresholds of scala-tympani electrodes.

4. The auditory prosthesis of claim 1, wherein said prosthesis generates a larger number of independent information channels compared to conventional cochlear implant devices.

5. The auditory prosthesis of claim 1, wherein said intra-neural electrodes stimulate intra-modiolar fibers that travel in fascicles grouped by cochlear region.

6. The auditory prosthesis of claim 1, wherein said prosthesis stimulates the inferior colliculus.

7. The auditory prosthesis of claim 6, wherein said prosthesis stimulates apical regions of said inferior colliculus.

8. The auditory prosthesis of claim 1, wherein said electrodes directly contact fibers originating from the spiral ganglion.

9. The auditory prosthesis of claim 1, wherein said intra-neural electrodes comprise an array of electrodes.

10. The auditory prosthesis of claim 9, wherein said array of electrodes comprises an array of 16 sites spaced in 100 μm intervals along a single shank.

11. The auditory prosthesis of claim 10, wherein said probe is configured to penetrate the osseous spiral lamina.

12. A method of inserting an auditory prosthesis, wherein said auditory prosthesis accesses the auditory nerve from a lateral approach, wherein said lateral approach comprises enlarging the round window and inserting a stimulating array of said prosthesis through a small hole made in the osseous spiral lamina.

13. The method of claim 12, wherein said stimulating array comprise an array of electrodes.

14. The auditory prosthesis of claim 13, wherein said array of electrodes comprises an array of 16 sites spaced in 100 μm intervals along a single shank.
